SEQUENCE LISTING (8 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGDGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGNGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 5 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGQGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 6 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGAGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 7 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGGGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 8 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGSGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2018/0163160 A1
(43) Pub. Date: Jun. 14, 2018

(54) STORAGE-STABLE LIQUID DETERGENT OR CLEANING AGENT CONTAINING PROTEASE AND LIPASE

(71) Applicant: BASF SE, Ludwigshafen (DE)

(72) Inventors: Susanne Wieland, Zons/Dormagen (DE); Karl-Heinz Maurer, Erkrath (DE); Timothy O'Connell, Duesseldorf (DE)

(73) Assignee: BASF SE, Ludwigshafen (DE)

(21) Appl. No.: 15/819,901

(22) Filed: Nov. 21, 2017

Related U.S. Application Data
(63) Continuation of application No. 13/674,170, filed on Nov. 12, 2012, now abandoned.
Application No. 13/674,170 is a continuation of application No. PCT/EP2011/057261, filed on May 6, 2011.

(30) Foreign Application Priority Data
May 12, 2010 (DE) 102010028951.5

(51) Int. Cl. C11D 3/386; C12N 9/54

(52) U.S. Cl. CPC C11D 3/38618; C12N 9/54; C11D 3/38681; C11D 3/38627

(57) ABSTRACT

The object is to improve the storage stability of a liquid washing or cleaning agent comprising a protease and lipase with regard to lipolytic activity. This is achieved through the use of a protease comprising an amino acid sequence that is at least 80% identical to the amino acid sequence set out in SEQ ID NO. 1 and that has the amino acid glutamic acid (E) or aspartic acid (D) or the amino acid asparagine (N) or glutamine (Q) or the amino acid alanine (A) or glycine (G) or serine (S) at position 99 in the sequence corresponding to SEQ ID NO. 1.

### FIELD OF THE INVENTION

The present invention generally relates to liquid washing and cleaning agents, and more particularly relates to liquid enzyme-containing washing and cleaning agents containing defined proteases in combination with a lipase, and it also proposes methods in which such agents are used. The invention further relates to uses of defined proteases in liquid washing or cleaning agents containing a lipase.

### BACKGROUND OF THE INVENTION

Proteases of the subtilisin type are preferably used for washing and cleaning agents. The proteases used in the washing or cleaning agents known from the prior art either derive originally from microorganisms, for example of the Bacillus, Streptomyces, Humicola or Pseudomonas species, and/or are produced by known biotechnological methods using suitable microorganisms, for example using transgenic expression hosts of the Bacillus species or using filamentous fungi.

Modern liquid washing agents in particular increasingly contain further enzymes, among them in particular lipases. A lipase is an enzyme that catalyzes the hydrolysis of ester bonds in lipid substrates, in particular in fats and oils. Lipases are therefore a group of esterases. Lipases are generally versatile enzymes that accept a large number of substrates, for example aliphatic, alicyclic, bicyclic and aromatic esters, thioesters and activated amines. Lipases counteract fat residues in the washing process, catalyzing their hydrolysis (lipolysis). Lipases having broad substrate spectra are used in particular where non-homogeneous raw materials or mixtures of substrates have to be reacted, thus for example in washing and cleaning agents, as stains can consist of variously structured fats and oils.

The world patent application WO 95/23221 discloses proteases and protease variants of the subtilisin type from Bacillus lentus DSM 5483, which are suitable for use in washing or cleaning agents. Among these proteases is one that can exhibit an amino acid exchange R99E, A, S or G. It is also disclosed that the washing agents can contain further enzymes, including a lipase. The washing agents can be solid or liquid. However, a liquid washing agent containing a lipase in combination with a protease having the amino acid glutamic acid (E) or aspartic acid (D) or the amino acid asparagine (N) or glutamine (Q) or the amino acid alanine (A) or glycine (G) or serine (S) at position 99 does not directly and unambiguously follow from this publication. The same is true of the European patent application EP 1921147.

A disadvantage of liquid washing and cleaning agents of the prior art containing protease and lipase is that they are not adequately stable in storage and thus lose a considerable measure of lipolytic and/or proteolytic activity, in particular lipolytic activity, after even a short time. The presence of protease frequently leads to the loss of lipolytic activity, since the protease inactivates the lipase. The cleaning efficiency of the washing or cleaning agent is then no longer optimal.

The object of the present invention is to overcome the cited disadvantage and to provide liquid washing or cleaning agents containing protease and lipase that have an adequate or improved storage stability, in particular with regard to their lipolytic activity.

The invention therefore provides a liquid washing or cleaning agent comprising
(a1) a protease comprising an amino acid sequence that is at least 80% identical to the amino acid sequence set out in SEQ lD NO. 1 and that has the amino acid glutamic acid (E) or aspartic acid (D) at position 99 in the sequence corresponding to SEQ ID NO. 1, or
(a2) a protease comprising an amino acid sequence that is at least 80% identical to the amino acid sequence set out in SEQ ID NO. 1 and that has the amino acid asparagine (N) or glutamine (Q) at position 99 in the sequence corresponding to SEQ ID NO. 1, or
(a3) a protease comprising an amino acid sequence that is at least 80% identical to the amino acid sequence set out in SEQ ID NO. 1 and that has the amino acid alanine (A) or glycine (G) or serine (S) at position 99 in the sequence corresponding to SEQ ID NO. 1, and
(b) a lipase.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A liquid washing or cleaning agent comprising a protease comprising an amino acid sequence that is at least 80% identical to the amino acid sequence set out in SEQ ID NO. 1 and that has the amino acid glutamic acid (E) or aspartic acid (D) at position 99 in the sequence corresponding to SEQ ID NO. 1, or a protease comprising an amino acid sequence that is at least 80% identical to the amino acid sequence set out in SEQ ID NO. 1 and that has the amino acid asparagine (N) or glutamine (Q) at position 99 in the sequence corresponding to SEQ ID NO. 1, or a protease comprising an amino acid sequence that is at least 80% identical to the amino acid sequence set out in SEQ ID NO. 1 and that has the amino acid alanine (A) or glycine (G) or serine (S) at position 99 in the sequence corresponding to SEQ ID NO. 1, and a lipase.

A liquid washing or cleaning agent comprising a protease selected from the group consisting of: protease comprising an amino acid sequence corresponding to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8; protease comprising an amino acid sequence that is modified in at least one position in comparison to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, the modification in the sequence corresponding to SEQ ID NO. 1 being selected from the group consisting of: threonine at position 3 (3T), isoleucine at position 4 (4I), alanine, threonine or arginine at position 61 (61A, 61T or 61R), aspartic acid or glutamic acid at position 154 (154D or 154E), proline at position 188 (188P), methionine at position 193 (193M), isoleucine at position 199 (199I), aspartic acid, glutamic acid or glycine at position 211 (211D, 211E or 211G), combinations of amino acids (i) to (viii); a lipase.

Use of a protease comprising an amino acid sequence that is at least 80% identical to the amino acid sequence set out in SEQ ID NO. 1 and that has the amino acid glutamic acid (E) or aspartic acid (D) at position 99 in the sequence corresponding to SEQ ID NO. 1, or comprising an amino acid sequence that is at least 80% identical to the amino acid sequence set out in SEQ ID NO. 1 and that has the amino acid asparagine (N) or glutamine (Q) at position 99 in the sequence corresponding to SEQ ID NO. 1, or comprising an amino acid sequence that is at least 80% identical to the amino acid sequence set out in SEQ ID NO. 1 and that has the amino acid alanine (A) or glycine (G) or serine (S) at position 99 in the sequence corresponding to SEQ ID NO. 1, to provide a proteolytic activity in a liquid washing or cleaning agent comprising a lipase.

Use of a protease selected from the group consisting of protease comprising an amino acid sequence corresponding to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8; protease comprising an amino acid sequence that is modified in at least one position in comparison to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, the modification in the sequence corresponding to SEQ ID NO. 1 being selected from the group consisting of: threonine at position 3 (3T), isoleucine at position 4 (4I), alanine, threonine or arginine at position 61 (61A, 61T or 61R), aspartic acid or glutamic acid at position 154 (154D or 154E), proline at position 188 (188P), methionine at position 193 (193M), isoleucine at position 199 (199I), aspartic acid, glutamic acid or glycine at position 211 (211D, 211E or 211G), combinations of amino acids (i) to (viii); to provide a proteolytic activity in a liquid washing or cleaning agent comprising a lipase.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly it has been found that a liquid washing or cleaning agent containing the combination of such a protease with a lipase is advantageously stable in storage. In particular it exhibits a higher lipolytic activity after storage in comparison to a washing or cleaning agent differing from an agent according to the invention only in terms of the protease present in each agent, the protease being present in the agents for comparison at the start of storage in the same concentration, relative to active enzyme. A protease provided within the context of the present invention therefore leads to a reduced inactivation of the lipase. The reduced inactivation of lipase by the protease provided within the context of the present invention is not based on an inadequate protease activity, however.

In this regard an agent according to the invention preferably continues to exhibit a good, in particular an advantageous cleaning efficiency in respect of protease-sensitive stains. Such a cleaning efficiency in respect of at least one protease-sensitive stain occurs in particular even at low temperatures, for example between 10° C. and 50° C., preferably between 10° C. and 40° C. or between 20° C. and 40° C. Such an agent therefore makes it possible to achieve a satisfactory or improved removal of at least one, preferably a plurality of protease-sensitive stains on textiles and/or hard surfaces, for example crockery.

With regard to the world patent application WO 95/23221 mentioned at the start, the present invention is a particularly advantageous choice that leads to the provision of a powerful and stable liquid washing agent, in particular with regard to the proteolytic and/or lipolytic activity of the agent or the residual activity of the agent after storage.

Within the context of the invention, cleaning efficiency is understood to mean the lightening efficiency on one or more stains, in particular laundry stains. Examples of such stains are blood-milk/ink on cotton, whole egg/pigment on cotton, chocolate-milk/ink on cotton, groundnut oil-pigment/ink on polyester/cotton, grass on cotton or cocoa on cotton, in particular of the type mentioned further below. Within the context of the invention both the washing or cleaning agent comprising the protease and lipase or the washing or cleaning liquor formed by this agent and the protease or lipase itself has a cleaning efficiency in its own right. The cleaning efficiency of the enzymes thus contributes to the cleaning efficiency of the agent or of the washing or cleaning liquor formed by the agent. The cleaning efficiency is preferably determined in the manner described further below.

A washing or cleaning liquor is understood to be the working solution containing the washing or cleaning agent, which acts on textiles or fabric (washing liquor) or on hard surfaces (cleaning liquor) and thus comes into contact with the stains present on textiles or fabrics or on hard surfaces. The washing or cleaning liquor conventionally forms when the washing or cleaning process begins and the washing or cleaning agent is diluted with water, for example in a washing machine or in another suitable vessel.

Storage stability within the meaning of the invention exists if after storage a washing or cleaning agent according to the invention has a higher lipase activity in comparison to a control composition that differs from the washing or cleaning agent according to the invention only in terms of the protease contained in the control composition. After storage, a washing or cleaning agent according to the invention therefore has a higher residual lipase activity in comparison to the control. Therefore at the start of storage both of the agents for comparison have the same amount or concentration of lipase and/or the same lipolytic starting activity. Furthermore, at the start of storage the protease is present in both agents in the same concentration, relative to active enzyme, and both agents are treated in the same way, in particular as regards the storage conditions and the determination of enzyme activity. In increasing order of preference, storage takes place for at least 24 hours, 48 hours, 72 hours, 5 days, 1 week, 2 weeks, 3 weeks or 4 weeks. Storage also preferably takes place at a temperature of 20° C., 25° C. or 30° C., particularly preferably at 30° C.

In this regard the enzyme activity can be determined in the customary technical manner, adapted to the particular enzyme type. Methods of determining activity are familiar to the person skilled in the art in the field of enzyme technology and are routinely used by him. Methods of determining protease activity are disclosed for example in Tenside, volume 7 (1970), p. 125-132. The proteolytic activity can moreover be determined from the release of the chromophore para-nitroaniline (pNA) from the substrate suc-L-Ala-L-Ala-L-Pro-L-Phe-p-nitroanilide (suc-AAPF-pNA). The protease cleaves the substrate and releases pNA. The release of pNA gives rise to an increase in extinction at 410 nm, the time course of which is a measure of enzymatic activity (cf. Del Mar et al., 1979). The measurement takes place at a temperature of 25° C., at pH 8.6 and at a wavelength of 410 nm. The measuring time is 5 min, with a measuring interval of 20 s to 60 s. The protease activity is preferably stated in PE (protease units).

Lipase activity is determined in the customary technical manner, and preferably as described in Bruno Stellmach, “Bestimmungsmethoden Enzyme für Pharmazie, Lebensmittelchemie, Technik, Biochemie, Biologie, Medizin” (Steinkopff Verlag Darmstadt, 1988, p. 172 ff). Here lipase-containing samples are added to an olive oil emulsion in water containing emulsifier and incubated at 30° C. and pH 9.0. Fatty acids are released in the process. These are titrated continuously with 0.01 N sodium hydroxide solution for 20 min with an auto-titrater in such a way that the pH remains constant (pH-stat titration). The lipase activity is determined from the sodium hydroxide consumption by reference to a reference lipase sample.

The protein concentration can be determined using known methods, for example the BCA method (bicinchoninic acid; 2,2′-bichinolyl-4,4′-dicarboxylic acid) or the Biuret method (A. G. Gornall, C. S. Bardawill and M. M. David, J. Biol. Chem., 177 (1948), p. 751-766). The active protein concentration can be determined by titrating the active centers using a suitable irreversible inhibitor (for proteases for example phenylmethylsulfonyl fluoride (PMSF)) and determining the residual activity (cf. M. Bender et al., J. Am. Chem. Soc. 88, 24 (1966), p. 5890-5913). The active protein content of a lipase preparation can be established in this regard by active-site titration of the lipase preparation using methyl p-nitrophenyl-n-hexylphosphonate as an inhibitor. Here different concentrations of the enzyme in an appropriate buffer system are provided with an excess of inhibitor and the amounts of p-nitrophenolate that are released are determined by spectrophotometry at a wavelength of 400 nm (in this regard cf. also Rotticci et al.; “An active-site titration method for lipases”; Biochim. Biophys. Acta 1483(1), p. 132-140). The above methods of determination are preferred within the context of the present invention.

The existence of an enzyme stabilization within the meaning of the present invention is particularly preferably established as stated above using a protease- and lipase-containing liquid washing or cleaning agent that has been stored for four weeks at a temperature of 30° C., the proteolytic activity being determined from the release of the chromophore para-nitroaniline (pNA) from the substrate suc-AAPF-pNA and the lipolytic activity being determined as described above.

The protease contained in a washing or cleaning agent according to the invention encompasses an amino acid sequence that is at least 80% identical to the amino acid sequence set out in SEQ ID NO. 1 and that has the amino acid glutamic acid (E) or aspartic acid (D) or the amino acid asparagine (N) or glutamine (Q) or the amino acid alanine (A) or glycine (G) or serine (S) at position 99 in the sequence corresponding to SEQ ID NO. 1. In increasing order of preference the amino acid sequence is at least 81%, 82%, 83%, 84%, 85%, 86%, 87%, 88%, 89%, 90%, 91%, 92%, 93%, 94%, 95%, 96%, 97%, 98% and most particularly preferably 99% identical to the amino acid sequence set out in SEQ ID NO. 1. SEQ ID NO. 1 is the sequence of the mature alkaline protease from Bacillus lentus DSM 5483, which is disclosed in the world patent application WO 92/21760, and to the disclosure of which reference is expressly made here.

It has been found according to the invention that by adding such a protease to a liquid washing or cleaning agent containing a lipase, a particularly stable liquid washing agent is obtained, in particular with regard to its remaining lipolytic activity after storage, in particular after a storage period of 1 to 8 weeks, 1 to 6 weeks, 1.5 to 5 weeks and particularly preferably after 4 weeks.

A protease contained in a washing or cleaning agent according to the invention has a proteolytic activity, which means that it is capable of hydrolyzing peptide bonds of a polypeptide or protein. It is therefore an enzyme that catalyzes the hydrolysis of peptide bonds and is thus capable of cleaving peptides or proteins. It is in particular a subtilase and particularly preferably a subtilisin.

Lipases that can be prepared according to the invention are for example the lipases obtainable originally from Humicola lanuginosa (Thermomyces lanuginosus) or the further developments thereof, in particular those with the amino acid exchange D96L. They are sold for example by Novozymes under the trade names Lipolase®, Lipolase®Ultra, LipoPrime®, Lipozyme® and Lipex®. A further lipase that can be used is obtainable from Novozymes under the trade name Lipoclean®. Furthermore, the cutinases that were originally isolated from Fusarium solani pisi and Humicola insolens can also be used, for example. Lipases that can likewise be used are obtainable from Amano under the names Lipase CE®, Lipase P®, for example Lipase PS®, Lipase B®, or Lipase CES®, Lipase AKG®, Bacillus sp. Lipase®, Lipase AP®, Lipase M-AP® and Lipase AML®. Lipases or cutinases from Genencor whose starting enzymes were originally isolated from Pseudomonas mendocina and Fusarium solanii, for example, can be used. Further important commercial products that can be mentioned are the preparations M1 Lipase® and Lipomax® originally sold by Gist-Brocades and the enzymes sold by Meito Sangyo KK, Japan, under the names Lipase MY-30®, Lipase OF® and Lipase PL®, also the product Lumafast® from Genencor. Particularly preferred lipases are disclosed in the laid-open world patent applications WO 92/05249, WO 00/60063, WO 2007/087508 and WO 2007/087503, to the disclosure of which reference is therefore expressly made or the relevant disclosure content of which is therefore expressly included in the present patent application.

In a further embodiment of the invention the washing or cleaning agent has the characterizing feature that the protease additionally has at least one of the following amino acids in the sequence corresponding to SEQ ID NO. 1:
(a) threonine at position 3 (3T),
(b) isoleucine at position 4 (4I),
(c) alanine, threonine or arginine at position 61 (61A, 61T or 61R),
(d) aspartic acid or glutamic acid at position 154 (154D or 154E),
(e) proline at position 188 (188P),
(f) methionine at position 193 (193M),
(g) isoleucine at position 199 (199I),
(h) aspartic acid, glutamic acid or glycine at position 211 (211D, 211E or 211G),
(i) combinations of amino acids (a) to (h).

In addition to one of the specified amino acids at position 99 the protease therefore has one or more of the aforementioned amino acids at the corresponding positions. These amino acids can bring about further advantageous properties and/or further strengthen existing properties. In particular, the aforementioned amino acids bring about an increase in the proteolytic activity and/or stability of the protease in a liquid washing or cleaning agent or in the washing liquor formed by this washing or cleaning agent. By adding such a protease to a liquid washing or cleaning agent containing a lipase, a particularly stable liquid washing agent is likewise obtained, in particular with regard to its remaining lipolytic activity after storage but preferably also with regard to its remaining proteolytic activity after storage, in particular after a storage period of 1 to 8 weeks, 1 to 6 weeks, 1.5 to 5 weeks and particularly preferably after 4 weeks. Such an agent moreover demonstrates improved cleaning efficiencies on protease- and/or lipase-sensitive stains.

The amino acid positions are defined by aligning the amino acid sequence of the protease to be used with the amino acid sequence of the protease from Bacillus lentus, as set out in SEQ ID NO. 1. As the protease from Bacillus lentus is an important reference molecule in the prior art for describing proteases and amino acid changes, it is advantageous to refer to the sequence of the protease from Bacillus lentus (SEQ ID NO. 1) in the assignment of amino acid positions. Furthermore, the sequence is based on the mature protein. This assignment should also be used in particular if the amino acid sequence of the protease to be used encompasses a higher number of amino acid residues than the protease from Bacillus lentus corresponding to SEQ ID NO. 1. Starting from the specified positions in the amino acid sequence of the protease from Bacillus lentus, the amino acid positions in a protease for use according to the invention are those assigned to precisely those positions in an alignment.

Particularly advantageous positions in addition to position 99 are thus positions 3, 4, 61, 154, 188, 193, 199 and 211, to be assigned in an alignment with SEQ ID NO. 1 and hence in the sequence corresponding to SEQ ID NO. 1. The following amino acid residues are in the cited positions in the wild-type molecule of the protease from Bacillus lentus: S3, V4, G61, S154, A188, V193, V199, and L211. The amino acids 3T, 4I, 61A, 154D, 154E, 211D, 211G and 211E are particularly preferred, provided that the corresponding positions in a protease for use according to the invention have not already been occupied by one of these preferred amino acids. The exchanges 3T and 4I for example have a stabilizing effect on the molecule that leads to an improvement in the storage stability and cleaning efficiency of the protease and hence to an improved cleaning efficiency of a liquid washing or cleaning agent according to the invention containing this protease.

If one or more of the aforementioned amino acids are put in place at the corresponding position, further sequence variations from SEQ ID NO. 1 occur in addition to position 99, as SEQ ID NO.1 has a different amino acid in the corresponding position. Depending on the number of sequence variations from SEQ ID NO. 1, there are therefore different maximum identity values that a protease for use according to the invention can have in relation to SEQ ID NO. 1, even if it is to correspond to SEQ ID NO. 1 in all other amino acids. This fact must be taken into consideration in the individual case for every possible combination of the proposed amino acids and is moreover also dependent on the length of the amino acid sequence of the protease. For example, the maximum identity with one, two, three, four, five, six, seven, eight or nine sequence changes is 99.63%, 99.26%, 98.88%, 98.51%, 98.14%, 97.77%, 97.40%, 97.03% or 96.65% respectively with a 269 amino acid-long amino acid sequence, or 99.64%, 99.27%, 98.91%, 98.55%, 98.18%, 97.82%, 97.45%, 97.09% or 96.73% respectively with a 275 amino acid-long amino acid sequence.

The identity of nucleic acid or amino acid sequences is determined by means of a sequence comparison. This type of comparison is performed by assigning similar sequences in the nucleotide sequences or amino acid sequences to one another. This sequence comparison is preferably based on the BLAST algorithm, which is established in the prior art and is conventionally used (cf. for example Altschul, S. F., Gish, W., Miller, W., Myers, E. W. & Lipman, D. J. (1990) “Basic local alignment search tool”, J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, p.3389-3402) and which takes place in principle by assigning similar sequences of nucleotides or amino acids in the nucleic acid or amino acid sequences to one another. A tabular assignment of the relevant positions is known as an alignment. Another algorithm that is available in the prior art is the FASTA algorithm. Sequence comparisons (alignments), in particular multiple sequence comparisons, are conventionally created with computer programs. For example, the Clustal series (cf. for example Chenna et al. (2003): Multiple sequence alignment with the Clustal series of programs. Nucleic Acid Research 31, 3497-3500), T-Coffee (cf. for example Notredame et al. (2000): T-Coffee: A novel method for multiple sequence alignments. J. Mol. Biol. 302, 205-217) or programs based on these programs or algorithms are frequently used. Within the context of the present invention, sequence comparisons and alignments are preferably created with the computer program Vector NTI® Suite 10.3 (Invitrogen Corporation, 1600 Faraday Avenue, Carlsbad, California, USA) with the predefined default parameters.

Such a comparison allows a statement to be made on the similarity to one another of the compared sequences. This is conventionally stated as the percentage identity, in other words the proportion of identical nucleotides or amino acid residues at the same positions or at positions corresponding to one another in an alignment. In amino acid sequences the broader concept of homology also includes conserved amino acid exchanges, in other words amino acids with similar properties, as these usually perform similar activities or functions within the protein. Therefore the similarity of the compared sequences can also be stated as the percentage homology or percentage similarity. Identity and/or homology data can refer to entire polypeptides or genes or only to individual areas. Homologous or identical areas of different nucleic acid or amino acid sequences are therefore defined by matches in the sequences. They often have identical or similar functions. They can be small and encompass only a few nucleotides or amino acids. Such small areas often perform essential functions for the overall activity of the protein. It can therefore be useful to relate sequence matches only to individual, possibly small areas. Unless otherwise specified, however, identity or homology data in the present application refers to the total length of the specified nucleic acid or amino acid sequence.

In a further embodiment of the subject-matter of this invention the washing or cleaning agent has the characterizing feature that the protease encompasses an amino acid sequence that is identical to the amino acid sequence set out in SEQ ID NO. 1 as stated above and that is obtained or is obtainable from a protease corresponding to SEQ ID NO. 1 by single or multiple conservative amino acid substitution, the protease at position 99 still having one of the amino acids provided for this position as described above. The term “conservative amino acid substitution” denotes the exchange (substitution) of one amino acid residue for another amino acid residue, wherein this exchange does not lead to a change in the polarity or charge at the position of the exchanged amino acid, for example the exchange of one non-polar amino acid residue for another non-polar amino acid residue. Conservative amino acid substitutions within the context of the invention encompass for example G=A=S, I=V=L=M, D=E, N=Q, K=R, Y=F, S=T, G=A=I=V=L=M=Y=F=W=P=S=T.

In a further embodiment of the invention a washing or cleaning agent according to the invention has the further characterizing feature that its cleaning efficiency corresponds to at least that of a washing or cleaning agent containing a protease that encompasses an amino acid sequence corresponding to the amino acid sequence set out in SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, particularly preferably in SEQ ID NO. 2. The cleaning efficiency is determined in a washing system containing a lipase-containing washing agent in a dose of between 2.0 and 9.0 grams per liter of washing liquor together with the protease, wherein the proteases to be compared are used in identical concentrations (relative to active protein) and the cleaning efficiency is determined in relation to one or more of the stains blood-milk/ink on cotton, whole egg/pigment (whole egg/carbon black) on cotton, groundnut oil-pigment/ink on polyester/cotton and grass on cotton, in particular in relation to one or more of the stains
blood-milk/ink on cotton: product no. C-05 obtainable from CFT (Center For Testmaterials) B. V. Vlaardingen, Netherlands
whole egg/pigment (whole egg/carbon black) on cotton: product no. 10N obtainable from wfk Testgewebe GmbH; Brüggen-Bracht, Germany, or product C-S-37 obtainable from CFT (Center For Testmaterials) B.V. Vlaardingen, Netherlands
groundnut oil-pigment/ink on polyester/cotton: product no. PC-10 obtainable from CFT (Center For Testmaterials) B. V. Vlaardingen, Netherlands
grass on cotton: product no. 164 obtainable from Eidgenössische Material-und Prüfanstalt (EMPA) Testmaterialien AG, St. Gallen, Switzerland
   by measuring the whiteness of the washed textiles, the washing process takes place for at least 30 minutes, optionally for 60 minutes, at a temperature of 20° C. and the water has a water hardness of between 15.5 and 16.5° (German hardness).

The washing agent for the washing system is a liquid washing agent having the following composition (all figures in percentage by weight): 0.3-0.5% xanthan gum, 0.2-0.4% antifoaming agent, 6-7% glycerol, 0.3-0.5% ethanol, 4-7% FAEOS (fatty alcohol ether sulfate), 24-28% non-ionic surfactants, 1% boric acid, 1-2% sodium citrate (dihydrate), 2-4% sodium carbonate, 14-16% coconut fatty acids, 0.5% HEDP (1-hydroxyethane-(1,1-diphosphonic acid)), 0-0.4% PVP (polyvinylpyrrolidone), 0-0.05% optical brightener, 0-0.001% dye, 0.0001-0.06% lipase (active protein), preferably 0.01-3% Lipex 100L (lipase preparation from Novozymes, active substance content 1.9%), remainder demineralized water. The protease is used in the washing agent in a concentration from 0.001 to 0.1%, preferably from 0.01 to 0.06%, relative to active protein. The dose of liquid washing agent is between 2.0 and 9.0, preferably between 3.0 and 8.2, between 4.0 and 7.5 and particularly preferably 4.7 grams per liter of washing liquor. Washing takes place in a pH range between pH 8 and pH 10.5, preferably between pH 8 and pH 9. Neither the protease activity nor the lipase activity in the washing liquor is zero at the start of washing.

The whiteness, i.e. the lightening of the stains, as a measure of cleaning efficiency is determined by optical measuring methods, preferably by photometry. A suitable instrument for this purpose is the Minolta CM508d spectrometer, for example. The instruments used for the measurement are conventionally calibrated in advance with a white standard, preferably a white standard supplied with the instrument.

In a further embodiment of the invention a washing or cleaning agent according to the invention has the further characterizing feature that its storage stability corresponds to at least that of a washing or cleaning agent containing a protease that encompasses an amino acid sequence corresponding to the amino acid sequence set out in SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, particularly preferably in SEQ ID NO. 2. Such a storage stability exists if after being stored for 4 weeks at 30° C. the washing or cleaning agent according to the invention has a lipase activity that is the same as or higher than the washing or cleaning agent to be used for comparison, the agent according to the invention differing from the washing or cleaning agent to be used for comparison only in terms of the protease it contains.

The agent to be used for comparison is particularly preferably a liquid washing agent having the following composition (all figures in percentage by weight): 0.3-0.5% xanthan gum, 0.2-0.4% antifoaming agent, 6-7% glycerol, 0.3-0.5% ethanol, 4-7% FAEOS (fatty alcohol ether sulfate), 24-28% non-ionic surfactants, 1% boric acid, 1-2% sodium citrate (dihydrate), 2-4% sodium carbonate, 14-16% coconut fatty acids, 0.5% HEDP (1-hydroxyethane-(1,1-diphosphonic acid)), 0-0.4% PVP (polyvinylpyrrolidone), 0-0.05% optical brightener, 0-0.001% dye, 0.0001-0.06% lipase (active protein), preferably 0.01-3% Lipex 100L (lipase preparation from Novozymes, active substance content 1.9%), remainder demineralized water. The protease is used in the washing agent in a concentration from 0.001 to 0.1%, preferably from 0.01 to 0.06%, relative to active protein.

At the start of storage both agents to be compared have the same lipolytic starting activity and contain the protease in the same concentration relative to active enzyme, and both agents are treated in the same way. The proteolytic activity in the agents is determined in each case from the release of the chromophore para-nitroaniline (pNA) from the substrate suc-AAPF-pNA, and their lipolytic activity is determined in each case in the manner described above. The starting activities for the protease and lipase in each agent are not equal to zero.

The use of lipases of equal activity and of proteases in equal concentrations, relative to active protein, ensures that even if there is any divergence in the ratio of active substance to total protein (the values of the specific activity), the actual enzymatic properties are compared.

Unless otherwise specified, within the context of the present invention reference is made in each case to the weight of the liquid washing agent, in other words the specified values relate to its weight.

Numerous proteases and in particular subtilisins are formed as pre-proteins, i.e. together with a pro-peptide and a signal peptide, the function of the signal peptide conventionally being to ensure the release of the protease from the cell producing it into the periplasma or the medium surrounding the cell and the pro-peptide conventionally being necessary for the correct folding of the protease. The signal peptide and the pro-peptide are generally the N-terminal part of the pre-protein. The signal peptide is cleaved from the rest of the protease under natural conditions by a signal peptidase. The correct final folding of the protease then takes place, supported by the pro-peptide. The protease is then in its active form and cleaves off the pro-peptide itself. Following cleavage of the pro-peptide, the then mature protease, in particular subtilisin, performs its catalytic activity without the originally present N-terminal amino acids. For technical applications in general and in particular within the context of the invention, the mature proteases, i.e. the enzymes processed after their production, are preferred over the pre-proteins. The proteases can moreover be modified by the cells producing them following production of the polypeptide chain, for example by the attachment of sugar molecules, formylations, aminations, etc. Such modifications are post-translational modifications and may, but do not have to, influence the function of the protease.

Furthermore, even the mature protease can be shortened at its N-terminal and/or C-terminal end, such that a shorter protease, or fragment, in comparison to SEQ ID NO. 1 or SEQ ID NO. 2 or SEQ ID NO. 3 or SEQ ID NO. 4 or SEQ ID NO. 5 or SEQ ID NO. 6 or SEQ ID NO. 7 or SEQ ID NO. 8 is contained in the washing or cleaning agent according to the invention. In this case all identity information relates to the area to which the individual fragment is assigned in a SEQ ID NO. 1 alignment. In every case, however, the individual fragment includes the position assigned to position 99 in an alignment with SEQ ID NO. 1 and has a corresponding amino acid at this position. It advantageously also includes one or more of the other positions described above and has corresponding amino acids there. Such a fragment is moreover proteolytically active. A further preferred fragment in this regard encompasses an amino acid sequence that over a length of at least 50 or at least 60, 70, 80, 90, 100, 110, 120, 130, 140, 150, 160, 170, 180, 190, 200, 210, 220, 230, 240, 250, 260, 265, 266, 267 or 268 successive amino acid positions matches SEQ ID NO. 1 or SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, taking into consideration the aforementioned amino acids for position 99 and optionally also for positions 3 and/or 4 and/or 61 and/or 154 and/or 188 and/or 193 and/or 199 and/or 211. The cleaning efficiency and/or storage stability of a liquid washing or cleaning agent according to the invention having such a fragment particularly preferably corresponds to at least that of a washing or cleaning agent containing a protease comprising an amino acid sequence corresponding to that set out in SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, determined in each case in the manner specified above.

The invention also provides a liquid washing or cleaning agent comprising
(a) a protease selected from the group consisting of
   a. protease comprising an amino acid sequence corresponding to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8;
   b. protease comprising an amino acid sequence that is modified in at least one position in comparison to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, the modification in the sequence corresponding to SEQ ID NO. 1 being selected from the group consisting of:
      i. threonine at position 3 (3T),
      ii. isoleucine at position 4 (4I),
      iii. alanine, threonine or arginine at position 61 (61A, 61T or 61R),
      iv. aspartic acid or glutamic acid at position 154 (154D or 154E),
      v. proline at position 188 (188P),
      vi. methionine at position 193 (193M),
      vii. isoleucine at position 199 (199I),
      viii. aspartic acid, glutamic acid or glycine at position 211 (211D, 211E or 211G),
      ix. combinations of amino acids (i) to (viii);
(b) a lipase.

These proteases are most particularly preferably used in a liquid washing or cleaning agent according to the invention. They are obtained starting from SEQ ID NO. 1 by the substitution of the amino acid arginine at position 99 with the amino acid glutamic acid (E) or aspartic acid (D) or the amino acid asparagine (N) or glutamine (Q) or the amino acid alanine (A) or glycine (G) or serine (S) in the sequence corresponding to SEQ ID NO. 1. These amino acid sequences are shown in the sequence protocol as SEQ ID NO. 2, SEQ ID NO. 3, SEQ ID NO. 4, SEQ ID NO. 5, SEQ ID NO. 6, SEQ ID NO. 7 and SEQ ID NO. 8. In addition to the amino acid provided for position 99 these proteases can moreover have one or more of the aforementioned amino acids in positions 3, 4, 61, 154, 188, 193, 199 and 211, for assignment in an alignment with SEQ ID NO. 1 and hence in the sequence corresponding to SEQ ID NO. 1. In these proteases too the cited amino acids for these positions give rise to further advantageous properties and/or strengthen already existing properties. In particular, they bring about an increase in the proteolytic activity and/or stability of the protease in a liquid washing or cleaning agent or in the washing liquor formed by this washing or cleaning agent. All above statements—where applicable—apply correspondingly to these particularly preferred proteases.

Agents according to the invention contain the protease and the lipase in a total amount from 1×10⁻⁸ to 5 wt. % each, relative to active protein. Each enzyme is preferably included in agents according to the invention in an amount from 0.0001 to 1% and more preferably from 0.0005 to 0.5%, 0.001 to 0.1% and particularly preferably from 0.001 to 0.06 wt. %, relative to active protein.

The protease and/or lipase can moreover be adsorbed on supporting materials and/or embedded in coating substances to protect them against premature inactivation. In the washing liquor, in other words under application conditions, the enzyme is then released and can develop its catalytic effect.

In a further embodiment of the invention the washing or cleaning agent has the characterizing feature that it additionally encompasses a component selected from
i. anionic and/or polyanionic substance, and/or
ii. cationic and/or polycationic substance, and/or
iii. substance containing hydroxyl and/or polyhydroxyl group(s).

It has been found that the addition of such substances further improves the cleaning efficiency of washing and cleaning agents, in particular of liquid washing or cleaning agents containing proteases and lipases, in particular those described above, in particular at comparatively low temperatures, in particular between 10° C. and 50° C., between 10° C. and 40° C., between 10° C. and 30° C. and/or between 20° C. and 40° C. When combined in particular with a protease for use according to the invention, a synergistic effect occurs, above all regarding the removal of at least one protease-sensitive stain, in particular one such as mentioned above.

The substances listed above under i. are anionic or polyanionic substances, in other words these substances bear at least one and preferably a plurality of negative charges. They are preferably a polymer having at least one negatively charged monomer, preferably having a plurality of negatively charged monomers. According to the invention this polymer is therefore preferably a negatively charged polymer. Polymers of organic acids or salts thereof, in particular polyacrylates and/or poly-sugar acids and/or polyacrylate copolymers and/or poly-sugar copolymers, are preferred for example. Further preferred compounds in this regard are polyacrylic sulfonates or polycarboxylates and salts thereof, copolymers or salts of copolymers.

Examples of substances that can particularly preferably be used are Acusol 587D (polyacrylic sulfonate; Rohm & Haas/Dow Chemical), Acusol 445N (polycarboxylate sodium salt; Rohm & Haas/Dow Chemical), Acusol 590 (polyacrylate copolymer; Rohm & Haas/Dow Chemical), Acusol 916 (polyacrylate sodium salt; Rohm & Haas/Dow Chemical), Sokalan CP42 (modified polycarboxylate sodium salt; BASF), Sokalan PA 30CL (polycarboxylate sodium salt; BASF), Dequest P 9000 (polymaleic acid; Thermphos), alginic acid, poly-2-acrylamido-2-methyl-1-propanesulfonic acid, poly-4-styrenesulfonic acid-co-maleic acid sodium salt, polyacrylamido-co-acrylic acid sodium salt, polymethacrylic acid sodium salt, polymethyl vinyl ether-alt-maleic acid or polyvinyl sulfonic acid sodium salt.

The substances listed under ii. are cationic or polycationic substances, in other words these substances bear at least one and preferably a plurality of positive charges. They are preferably a polymer having at least one positively charged monomer, preferably having a plurality of positively charged monomers. According to the invention this polymer is therefore preferably a positively charged polymer. Examples of compounds that are preferred in this regard are salts of polyamines, polyethyleneimines or copolymers thereof, salts of polyallylamines, salts of polydiallyldimethylammonium compounds or poly(acrylamide-co-diallyldimethylammonium) compounds.

The substances listed under iii. are substances having at least one hydroxyl and/or polyhydroxyl group and preferably a plurality of hydroxyl and/or polyhydroxyl groups. Polyvinyl alcohols, for example those available under the trade name Mowiol (Kremer Pigmente GmbH & Co. KG), are preferred for example in this regard.

It is expressly stated at this point that a specific substance can belong to one or more of the aforementioned groups i. to iii. It can for example be an anionic polymer having one or more hydroxyl and/or polyhydroxyl groups. Such a substance then belongs to groups i. and iii. Likewise a cationic polymer having one or more hydroxyl and/or polyhydroxyl groups belongs to groups ii. and iii.

Derivatives of the substances listed above as belonging to i., ii. or iii. can likewise be used within the context of the present invention. Within the meaning of the present application a derivative is understood to be a substance which, starting from one of the aforementioned substances, is chemically modified, for example by the conversion of a side chain or by covalent bonding of another compound to the substance. Such a compound can for example be low-molecular-weight compounds such as lipids or mono-, oligo- or polysaccharides or amines or amine compounds. The substance can moreover be glycosylated, hydrolyzed, oxidized, N-methylated, N-formylated or N-acetylated or can contain methyl, formyl, ethyl, acetyl, t-butyl, anisyl, benzyl, trifluoroacetyl, N-hydroxysuccinimide, t-butyloxycarbonyl, benzoyl, 4-methylbenzyl, thioanizyl, thiocresyl, benzyloxymethyl, 4-nitrophenyl, benzyloxycarbonyl, 2-nitrobenzoyl, 2-nitrophenylsulfenyl, 4-toluenesulfonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl, triphenylmethyl, 2,2,5,7,8-pentamethyl-chromane-6-sulfonyl. A derivative can likewise be understood to be the covalent or non-covalent bonding of the substance to a macromolecular support or a non-covalent inclusion in suitable macromolecular cage structures. Couplings with other macromolecular compounds, such as polyethylene glycol for instance, can also be undertaken. Further preferred chemical modifications are the modification of one or more of the chemical groups —COOH, —OH, ═NH, —NH₂—SH to —COOR, —OR, —NHR, —NR₂, —NHR, —NR, —SR; in which:
R is —CH═CH—R2, —C≡C—R2, —C(R2)═CH₂, —C(R2)═C(R3), —CH═NR2, —C(R2)═N—R3, a 4-7 C ring system with or without substitution, a 4-7 nitrogen heterocycle with or without substitution, or a C₂ to C₈ chain having 1 to 5 double or triple bonds with substitutions chosen from R1, R2, or R3, in which
R1 is H, —R, —NO₂, —CN, halide substituent, —N₃, —C₁₋₈ alkyl, —(CH₂)nCO₂R2, —C₂₋₈ alkenyl CO₂R2, —O(CH₂)nCO₂R2, —C(O)NR2R3, —P(O)(OR2)₂, alkyl-substituted tetrazol-5-yl, —(CH₂)nO(CH₂)n aryl, —NR2R3, —(CH₂)n OR2, —(CH₂)n SR2, —N(R2)C(O)R3, —S(O₂)NR2R3, —N(R2)S(O₂)R3, —(CHR2)n NR2R3, —C(O)R3, (CH₂)n N(R3)C(O)R3, —N(R2)CR2R3, substituted or unsubstituted (CH₂)n-cycloalkyl, substituted or unsubstituted (CH₂)n phenyl or cycle; in which n is a number greater than 1,
R2 is H, halide substituent, alkyl, haloalkyl, —(CH₂)n phenyl, —(CH₂)1-3-biphenyl, —(CH₂)1-4-Ph-N(SO₂—C₁₋₂ alkyl)₂, —CO(CHR1)n-OR1, —(CHR1)n heterocycle, —(CHR1)n-NH—CO—R1, —(CHR1)n-NH—SO₂R1, —(CHR1)n-Ph-N(SO₂—C₁₋₂ alkyl)₂, —(CHR1)n-C(O)(CHR1)—NHR1, —(CHR1)n-C(S)(CHR1)—NHR1, —(CH₂)nO(CH₂)nCH₃, —CF₃, —C₂-C₅ acyl, —(CHR1)nOH, —(CHR1)nCO₂R1, —(CHR1)n-O alkyl, —(CHR1)n-O—(CH₂)n-O alkyl, —(CHR1)n-S alkyl, —(CHR1)n-S(O) alkyl, —(CHR1)n-S(O₂) alkyl, —(CHR1)n-S(O₂)—NHR3, —(CHR3)n-N₃, —(CHR3)nNHR4, a C₂ to C₈-chain alkene chain having 1 to 5 double bonds, a C₂ to C₈-chain alkyne chain having 1 to 5 triple bonds, substituted or unsubstituted —(CHR3)n heterocycle, substituted or unsubstituted saturated or unsaturated —(CHR3)n cycloalkyl; in which n is a number greater than 1 and R1 and R3 can be the same or different;
R3 is H, —OH, —CN, substituted alkyl, —C₂ to C₈ alkenyl, substituted or unsubstituted cycloalkyl, N(R1)R2, saturated or unsaturated C₅ to C₇ heterocycle or heterobicycle of 4 to 7 C atoms, —NR1, —NR2, —NR1R2 consisting of a saturated or unsaturated heterocycle or a heterobicycle of 4 to 7 C atoms;
R4 is H, —(CH₂)nOH, —C(O)OR5, —C(O)SR5, —(CH₂)n C(O)NR6R7, —O—C(O)—O—R6, an amino acid or a peptide; in which n is a number between 0 and 4;
R5 is H,
R6 is —C(R7)—(CH₂)n-O—C(O)—R8, —(CH₂)n-C(R7)—O—C(O)R8, —(CH₂)n-C(R7)—O—C(O)—O—R8, or —C(R7)—(CH₂)n-O—C(O)—O—R8; in which n is a number between 0 and 4;
and
R7 and R8 are in each case H, alkyl, substituted alkyl, aryl, substituted aryl, alkenyl, substituted alkenyl, alkynyl, substituted alkynyl, heterocycle, substituted heterocycle, alkylaryl, substituted alkylaryl, cycloalkyl, substituted cycloalkyl, or CH₂CO₂ alkyl, in which R7 and R8 can be the same or different.

It is moreover possible according to the invention to use all possible combinations of the substances and/or derivatives named above as belonging to i., ii. or iii.

A liquid washing or cleaning agent according to the invention can be used as is or after dilution with water to clean textiles and/or hard surfaces. Such a dilution can easily be produced by diluting a measured amount of the agent in a further amount of water in specified weight ratios of agent to water and optionally shaking this dilution to ensure a uniform distribution of the agent in the water. Possible weight or volume ratios of the dilutions are from 1:0 agent:water to 1:10,000 or 1:20,000 agent:water, preferably from 1:10 to 1:2000 agent:water.

All liquid or free-flowing presentation forms can serve here as liquid washing or cleaning agents. “Free-flowing” within the meaning of the present application refers to agents that are pourable and that can have viscosities of up to several tens of thousands of mPas. The viscosity can be measured with conventional standard methods (for example Brookfield LVT-II viscometer at 20 rpm and 20° C., spindle 3) and is preferably in the range from 5 to 10,000 mPas. Preferred agents have viscosities from 10 to 8000 mPas, with values between 120 and 3000 mPas being particularly preferred. A liquid washing or cleaning agent within the context of the present invention can therefore be in the form of a gel or paste, it can take the form of a homogeneous solution or suspension, and can for example be sprayable or be formulated in other conventional presentation forms. The washing agents include all conceivable types of washing agent, in particular washing agents for textiles, carpets or natural fibers. They can also be intended for manual and/or automatic use. The washing agents additionally include washing auxiliary agents that are added to the actual washing agent during the manual or automatic textile washing process to achieve an additional effect. The cleaning agents include all agents likewise occurring in all specified presentation forms for cleaning and/or disinfecting hard surfaces, manual and automatic dishwashing agents, carpet cleaners, scouring agents, glass cleaners, toilet fresheners, etc. Finally textile pre- and aftertreatment agents are firstly agents that are brought into contact with the laundry item before it is actually washed, for example to partially dissolve stubborn stains, and secondly agents that in a subsequent step after the actual textile washing process impart further desirable properties to the laundry item, such as a pleasant feel, crease resistance or low static charge. The latter agents include inter alia fabric softeners. Disinfectants are for example hand disinfectants, surface disinfectants and instrument disinfectants, which can likewise occur in the specified presentation forms.

In a further preferred embodiment of the invention the washing or cleaning agent has the characterizing feature that it encompasses at least one further ingredient, in particular one selected from the group consisting of phosphonate, surfactant, builder, non-aqueous solvent, acid, water-soluble salt, thickening agent and combinations thereof.

Phosphonates are salts and organic compounds, in particular esters, of phosphonic acid. Salts are primary (M′H2PO3 or HP(O)(OH)(OM′)) and secondary (M′2HPO3 or HP(O)(OM′)2) phosphonates, in which M′ denotes a monovalent metal. These inorganic phosphonates are also known as primary or secondary phosphites. Inorganic phosphonates are formed for example by reacting phosphonic acid HP(O)(OH)2, in particular the stable tautomeric form of phosphorous acid, with one (primary) or two (secondary) equivalents of base, for example alkali metal hydroxide. Within the context of the present invention organic P-substituted phosphonates having a phosphorus-carbon bond (organophosphorus compounds) are preferred. Their general structure is R1P(O)(OR2)2, with R1 and/or R2=alkyl, aryl or H, in which the alkyl or aryl residues can have further substitutions or can bear further chemical groups. Organic P-substituted phosphonates are produced for example by the Michaelis-Arbusov reaction. Many of these phosphonates are soluble in water. Some technically important phosphonates also bear amino groups in the manner of NR—(CH2)x—PO(OH)2 (R=alkyl, aryl or H). Some of these aminophosphonates have structural similarities to complexing agents such as EDTA, NTA or DTPA and have a similar function. Particularly preferred phosphonates within the context of the present invention include in particular organophosphonates such as for example 1-hydroxyethane-1,1-diphosphonic acid (HEDP), amino tri(methylene phosphonic acid) (ATMP, also known as amino tris(methylene phosphonic acid) or nitrolotris(methylene phosphonic acid) (NTMP)), diethylenetriamine penta(methylene phosphonic acid) (DTPMP or DETPMP or DTPNT), ethylene diamine tetra(methylene phosphonic acid) (EDTMP, also known as ethylene diamine tetra(methylene phosphonic acid) and 2-phosphonobutane-1,2,4-tricarboxylic acid (PBS-AM, also known as 2-phosphonobutane-1,2,4-tricarboxylic acid or 3-carboxy-3-phosphonoadipic acid), which are mostly used in the form of their ammonium or alkali metal salts. Diethylenetriamine penta(methylene phosphonic acid) sodium is particularly preferred. Such a phosphonate is obtainable for example under the trade name Dequest® 2066 (Thermphos).

The phosphonate is preferably contained in the washing or cleaning agent in an amount from 0.01 to 2.5 wt. % and in increasing order of preference from 0.02 to 2 wt. %, from 0.03 to 1.5 wt. % and in particular from 0.05 to 1 wt. %.

Anionic, non-ionic, zwitterionic and/or amphoteric surfactants can be used as surfactant(s). From an application-oriented perspective, mixtures of anionic and non-ionic surfactants are preferred. The total surfactant content of the liquid washing or cleaning agent is preferably below 60 wt. % and particularly preferably below 45 wt. %, relative to the total liquid washing or cleaning agent.

Suitable non-ionic surfactants encompass alkoxylated fatty alcohols, alkoxylated fatty acid alkyl esters, fatty acid amides, alkoxylated fatty acid amides, polyhydroxy fatty acid amides, alkylphenol polyglycol ethers, amine oxides, alkyl polyglucosides and mixtures thereof.

Alkoxylated, advantageously ethoxylated, in particular primary alcohols having preferably 8 to 18 C atoms and on average 1 to 12 mol of ethylene oxide (EO) per mol of alcohol are preferably used as non-ionic surfactants, in which the alcohol residue can be linear or preferably methyl-branched in the 2-position or can contain linear and methyl-branched residues in the mixture, such as are conventionally present in oxoalcohol residues. However, alcohol ethoxylates having linear residues obtained from alcohols of native origin having 12 to 18 C atoms, for example from coconut, palm, tallow or oleyl alcohol, and on average 2 to 8 EO per mol of alcohol are preferred in particular. The preferred ethoxylated alcohols include, for example, C₁₂₋₁₄ alcohols having 3 EO, 4 EO or 7 EO, C₉₋₁₁ alcohol having 7 EO, C₁₃₋₁₅ alcohols having 3 EO, 5 EO, 7 EO or 8 EO, C₁₂₋₁₈ alcohols having 3 EO, 5 EO or 7 EO and mixtures thereof, such as mixtures of C₁₂₋₁₄ alcohol having 3 EO and C₁₂₋₁₈ alcohol having 7 EO. The specified degrees of ethoxylation are statistical averages which for an individual product can be a whole number or a fraction. Preferred alcohol ethoxylates have a narrow homolog distribution (narrow range ethoxylates, NRE). In addition to these non-ionic surfactants, fatty alcohols having more than 12 EO can also be used. Examples thereof are tallow fatty alcohol having 14 EO, 25 EO, 30 EO or 40 EO. Non-ionic surfactants containing EO and PO groups together in the molecule can also be used according to the invention. Also suitable are a mixture of a (highly) branched ethoxylated fatty alcohol and an unbranched ethoxylated fatty alcohol, such as for example a mixture of a C₁₆₋₁₈ fatty alcohol having 7 EO and 2-propyl heptanol having 7 EO. The washing, cleaning, aftertreatment or washing auxiliary agent preferably contains in particular a C₁₂₋₁₈ fatty alcohol having 7 EO or a C₁₃₋₁₅ oxoalcohol having 7 EO as the non-ionic surfactant.

The content of non-ionic surfactants in the washing or cleaning agent is preferably 3 to 40 wt. %, by preference 5 to 30 wt. % and in particular 7 to 20 wt. %, relative in each case to the total washing or cleaning agent.

In addition to the non-ionic surfactants the washing or cleaning agent can also contain anionic surfactants. Sulfonates, sulfates, soaps, alkyl phosphates, anionic silicone surfactants and mixtures thereof are preferably used as the anionic surfactant.

Suitable surfactants of the sulfonate type are preferably C₉₋₁₃ alkylbenzene sulfonates, olefin sulfonates, i.e. mixtures of alkene and hydroxyalkane sulfonates, and disulfonates, such as are obtained for example from C₁₂₋₁₈ monoolefins having a terminal or internal double bond by sulfonation with gaseous sulfur trioxide and subsequent alkaline or acid hydrolysis of the sulfonation products. Also suitable are C₁₂₋₁₈ alkane sulfonates and the esters of α-sulfo fatty acids (ester sulfonates), for example the α-sulfonated methyl esters of hydrogenated coconut, palm kernel or tallow fatty acids.

The alkali and in particular the sodium salts of the sulfuric acid semi-esters of C₁₂-C₁₈ fatty alcohols, for example of coconut fatty alcohol, tallow fatty alcohol, lauryl, myristyl, cetyl or stearyl alcohol, or of C₁₀-C₂₀ oxoalcohols and the semi-esters of secondary alcohols having these chain lengths are preferred as alk(en)yl sulfates. From a detergent perspective the C₁₂-C₁₆ alkyl sulfates and C₁₂-C₁₅ alkyl sulfates and C₁₄-C₁₅ alkyl sulfates are preferred. 2,3-Alkyl sulfates are also suitable anionic surfactants.

The sulfuric acid monoesters of the straight-chain or branched C₇₋₂₁ alcohols ethoxylated with 1 to 6 mol of ethylene oxide, such as 2-methyl-branched C₉₋₁₁ alcohols having on average 3.5 mol of ethylene oxide (EO) or C₁₂₋₁₈ fatty alcohols having 1 to 4 EO, are also suitable.

Soaps are also preferred anionic surfactants. Saturated and unsaturated fatty acid soaps are suitable, such as the salts of lauric acid, myristic acid, palmitic acid, stearic acid, (hydrogenated) erucic acid and docosanoic acid, and in particular soap mixtures derived from natural fatty acids, for example coconut, palm kernel, olive oil or tallow fatty acids.

The anionic surfactants including the soaps can be present in the form of their sodium, potassium or magnesium or ammonium salts. The anionic surfactants are preferably in the form of their sodium salts. Further preferred counterions for the anionic surfactants are also the protonated forms of choline, triethylamine or methylethylamine.

The content of anionic surfactants in a washing or cleaning agent can be 1 to 40 wt. %, preferably 5 to 30 wt. % and most particularly preferably 10 to 25 wt. %, relative in each case to the total washing or cleaning agent.

Examples of builders that can be included in the washing or cleaning agent are in particular silicates, aluminum silicates (in particular zeolites), carbonates, salts of organic di- and polycarboxylic acids and mixtures of these substances.

Organic builders that can be present in the washing or cleaning agent are for example the polycarboxylic acids that can be used in the form of their sodium salts, polycarboxylic acids being understood to be carboxylic acids bearing more than one acid function. These are for example citric acid, adipic acid, succinic acid, glutaric acid, malic acid, tartaric acid, maleic acid, fumaric acid, sugar acids, aminocarboxylic acids, nitrilotriacetic acid (NTA), methyl glycine diacetic acid (MGDA) and derivatives and mixtures thereof. Preferred salts are the salts of polycarboxylic acids such as citric acid, adipic acid, succinic acid, glutaric acid, tartaric acid, sugar acids and mixtures thereof.

Polymeric polycarboxylates are also suitable as builders. These are for example the alkali metal salts of polyacrylic acid or polymethacrylic acid, for example those having a relative molar mass of 600 to 750,000 g/mol.

Suitable polymers are in particular polyacrylates, which preferably have a molar mass of 1000 to 15,000 g/mol. Of this group, owing to their superior solubility, preference can in turn be given to the short-chain polyacrylates having molar masses of 1000 to 10,000 g/mol and particularly preferably 1000 to 5000 g/mol.

Also suitable are copolymeric polycarboxylates, in particular those of acrylic acid with methacrylic acid and of acrylic acid or methacrylic acid with maleic acid. To improve their solubility the polymers can also contain allyl sulfonic acids, such as allyloxybenzenesulfonic acid and methallyl sulfonic acid, as monomers.

However, soluble builders, such as for example citric acid, or acrylic polymers having a molar mass of 1000 to 5000 g/mol are preferably used in the liquid washing or cleaning agents.

Within the meaning of this publication the molar masses specified for the polymeric polycarboxylates are weight-average molar masses M_{w} of the individual acid form, which were determined in principle by gel permeation chromatography (GPC) using a UV detector. The measurement was carried out against an external polyacrylic acid standard, which because of its structural affinity to the polymers under investigation delivers realistic molar mass values. These figures differ markedly from the molar mass values obtained using polystyrene sulfonic acids as the standard. The molar masses measured against polystyrene sulfonic acids are generally significantly higher than the molar masses given in this publication.

Such organic builder substances can be included if desired in amounts of up to 40 wt. %, in particular up to 25 wt. % and preferably from 1 wt. % to 8 wt. %. Amounts close to the cited upper limit are preferably used in paste-form or liquid, in particular water-containing, agents.

The washing or cleaning agents according to the invention are liquid and contain preferably water as the main solvent. Non-aqueous solvents can additionally be added to the washing or cleaning agent. Suitable non-aqueous solvents encompass mono- or polyhydric alcohols, alkanolamines or glycol ethers, provided they are miscible with water in the specified concentration range. The solvents are preferably selected from ethanol, n-propanol, i-propanol, butanols, glycol, propanediol, butanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, diisopropylene glycol monomethyl ether, diisopropylene glycol monoethyl ether, methoxytriglycol, ethoxytriglycol, butoxytriglycol, 1-butoxyethoxy-2-propanol, 3-methyl-3-methoxybutanol, propylene glycol t-butyl ether, di-n-octyl ether and mixtures of these solvents. It is however preferable for the washing or cleaning agent to contain a polyol as the non-aqueous solvent. The polyol can in particular encompass glycerol, 1,2-propanediol, 1,3-propanediol, ethylene glycol, diethylene glycol and/or dipropylene glycol. The washing or cleaning agent preferably contains in particular a mixture of a polyol and a monohydric alcohol. Non-aqueous solvents can be used in the washing or cleaning agent in amounts of between 0.5 and 15 wt. %, but preferably below 12 wt. %.

To set a desired pH that is not established automatically by mixing the other components, the agents can contain system-compatible and environmentally compatible acids, in particular citric acid, acetic acid, tartaric acid, malic acid, lactic acid, glycolic acid, succinic acid, glutaric acid and/or adipic acid, but also mineral acids, in particular sulfuric acid, or bases, in particular ammonium or alkali hydroxides. Such pH regulators are included in the agents in amounts preferably not exceeding 20 wt. %, in particular from 1.2 wt. % to 17 wt. %.

An agent within the meaning of the invention can furthermore contain one or more water-soluble salts, which serve the purpose of viscosity adjustment for example. They can be inorganic and/or organic salts. Inorganic salts that can be used are preferably selected from the group comprising colorless water-soluble halides, sulfates, sulfites, carbonates, hydrogen carbonates, nitrates, nitrites, phosphates and/or oxides of alkali metals, alkaline-earth metals, aluminum and/or transition metals; ammonium salts can also be used. Halides and sulfates of alkali metals are particularly preferred; the inorganic salt is therefore preferably selected from the group comprising sodium chloride, potassium chloride, sodium sulfate, potassium sulfate and mixtures thereof. Organic salts that can be used are for example colorless water-soluble alkali metal, alkaline-earth metal, ammonium, aluminum and/or transition metal salts of carboxylic acids. The salts are preferably selected from the group comprising formate, acetate, propionate, citrate, malate, tartrate, succinate, malonate, oxalate, lactate and mixtures thereof.

An agent according to the invention can contain one or more thickening agents for thickening purposes. The thickening agent is preferably selected from the group comprising xanthan gum, guar gum, carrageenan, agar agar, gellan, pectin, carob seed meal and mixtures thereof. These compounds are effective thickening agents even in the presence of inorganic salts. In a particularly preferred embodiment the washing or cleaning agent contains xanthan gum as the thickening agent, as xanthan gum is an effective thickener even in the presence of high salt concentrations and prevents a macroscopic separation of the continuous phase. The thickening agent additionally stabilizes the continuous, low-surfactant phase and prevents a macroscopic phase separation.

Alternatively or in addition, (meth)acrylic acid (co)polymers can also be used as thickening agents. Suitable acrylic and methacrylic (co)polymers encompass for example the high-molecular-weight homopolymers of acrylic acid crosslinked with a polyalkenyl polyether, in particular an allyl ether of sucrose, pentaerythritol or propylene (INCI name in accordance with the International Dictionary of Cosmetic Ingredients published by the Cosmetic, Toiletry and Fragrance Association (CTFA): Carbomer), which are also classed as carboxyvinyl polymers. Such polyacrylic acids are obtainable inter alia under the trade names Polygel® and Carbopol®. Furthermore, the following acrylic acid copolymers for example are suitable: (i) copolymers of two or more monomers from the group of acrylic acid, methacrylic acid and their simple esters, preferably formed with C₁₋₄ alkanols (INCI Acrylates Copolymer), which are obtainable for example under the trade names Aculyn®, Acusol® or Tego® Polymer; (ii) crosslinked high-molecular-weight acrylic acid copolymers, including for instance the copolymers of C₁₀₋₃₀ alkyl acrylates with one or more monomers from the group of acrylic acid, methacrylic acid and their simple esters, preferably formed with C₁₄ alkanols, crosslinked with an allyl ether of sucrose or pentaerythritol (INCI Acrylates/C₁₀₋₃₀ Alkyl Acrylate Crosspolymer), obtainable for example under the trade name Carbopol®. Further suitable polymers are (meth)acrylic acid (co)polymers of the Sokalan® type.

It can be preferable for the washing or cleaning agent according to the invention to contain a (meth)acrylic acid (co)polymer in combination with a further thickening agent, preferably xanthan gum. The washing or cleaning agent can contain 0.05 to 1.5 wt. % and preferably 0.1 to 1 wt. %, relative in each case to the total washing or cleaning agent, of thickening agent. The amount of thickening agent used is dependent on the type of thickening agent and the desired degree of thickening.

Liquid or paste agents according to the invention in the form of solutions containing conventional solvents are generally produced by simply mixing the ingredients, which can be introduced into an automatic mixer in bulk or as a solution. Washing or cleaning agents according to the invention can exclusively contain a protease and a lipase as described. Alternatively they can also contain further hydrolytic enzymes or other enzymes in an appropriate concentration for the effectiveness of the agent. The invention therefore also provides agents that additionally encompass one or more further enzymes, wherein all enzymes established for these purposes in the prior art can in principle be used. All enzymes that can develop a catalytic activity in the agent according to the invention are preferably suitable as further enzymes, in particular a protease, amylase, cellulase, hemicellulase, mannanase, tannase, xylanase, xanthanase, xyloglucanase, β-glucosidase, pectinase, carrageenase, perhydrolase, oxidase, oxidoreductase or a lipase, and mixtures thereof. Further enzymes are advantageously each contained in the agent in a total amount from 1×10-8 to 5 wt. %, relative to active protein. Each enzyme is preferably included in agents according to the invention in an amount from 0.0001 to 1% and more preferably from 0.0005 to 0.5%, 0.001 to 0.1% and particularly preferably from 0.001 to 0.06 wt. %, relative to active protein. The enzymes particularly preferably demonstrate synergistic cleaning efficiencies in respect of specific stains or marks, in other words the enzymes contained in the agent composition are mutually supportive of one another in their cleaning efficiency. Such a synergy most particularly preferably exists between the protease included according to the invention and a further enzyme of an agent according to the invention, in particular between the cited protease and the lipase and/or an amylase and/or a mannanase and/or a cellulase and/or a pectinase. Synergistic effects can occur not only between different enzymes but also between one or more enzymes and other ingredients of the agent according to the invention.

The invention also provides the use of a washing or cleaning agent according to the invention for removing stains, in particular protease- and/or lipase-sensitive stains, on textiles or hard surfaces, i.e. for cleaning textiles or hard surfaces. By virtue in particular of the combination of protease and lipase they contain, agents according to the invention can advantageously be used for removing corresponding stains from textiles or hard surfaces. Embodiments of this subject-matter of the invention are for example manual washing, the manual removal of marks from textiles or hard surfaces, or use in conjunction with an automatic method. All data, subject-matters and embodiments described for washing or cleaning agents according to the invention can also be applied to this subject-matter of the invention. Therefore reference is expressly made here to the disclosure at the corresponding point, with the note that this disclosure also applies to the above use according to the invention.

The invention also provides a method for cleaning textiles or hard surfaces, wherein a washing or cleaning agent according to the invention is applied in at least one process step.

These include both manual and automatic methods, automatic methods being preferred because of their more accurate controllability in terms of for example the amounts used and contact periods. Methods for cleaning textiles generally have the characterizing feature that in a plurality of process steps various active cleaning substances are applied to the item to be cleaned and washed off after the contact period or that the item to be cleaned is treated with a washing agent or a solution or dilution of that agent by some other means. The same applies to methods for cleaning all materials other than textiles, in particular hard surfaces. All conceivable washing or cleaning methods can be enhanced in at least one of the process steps by the use of a washing or cleaning agent according to the invention and then constitute embodiments of the present invention. All data, subject-matters and embodiments described for washing or cleaning agents according to the invention can also be applied to this subject-matter of the invention. Therefore reference is expressly made here to the disclosure at the corresponding point, with the note that this disclosure also applies to the above methods according to the invention.

In a preferred embodiment the method has the characterizing feature that the lipase is present in the washing liquor in a concentration from 0.00075 to 0.03 wt. %, preferably from 0.003 to 0.027 wt. %, and/or the protease is present in the washing liquor in a concentration from 0.00075 to 0.03 wt. %, preferably from 0.003 to 0.027 wt. %. The concentration figures relate to the total washing liquor. In a further preferred embodiment the method has the characterizing feature that it is performed at a temperature between 10° C. and 50° C., preferably between 10° C. and 40° C. and particularly preferably between 20° C. and 40° C.

In accordance with the above embodiments, proteases used in agents according to the invention can advantageously be used in washing and cleaning agents according to the invention and in methods according to the invention, in particular washing and cleaning methods. They can also advantageously be used to provide a proteolytic activity in corresponding agents.

The invention therefore also provides the use of a protease
(a1) comprising an amino acid sequence that is at least 80% identical to the amino acid sequence set out in SEQ ID NO. 1 and that has the amino acid glutamic acid (E) or aspartic acid (D) at position 99 in the sequence corresponding to SEQ ID NO. 1, or
(a2) comprising an amino acid sequence that is at least 80% identical to the amino acid sequence set out in SEQ ID NO. 1 and that has the amino acid asparagine (N) or glutamine (Q) at position 99 in the sequence corresponding to SEQ ID NO. 1, or
(a3) comprising an amino acid sequence that is at least 80% identical to the amino acid sequence set out in SEQ ID NO. 1 and that has the amino acid alanine (A) or glycine (G) or serine (S) at position 99 in the sequence corresponding to SEQ ID NO. 1, to provide a proteolytic activity in a liquid washing or cleaning agent that also encompasses a lipase.

In a further embodiment this use has the characterizing feature that the protease additionally has at least one of the following amino acids in the sequence corresponding to SEQ ID NO. 1:
(a) threonine at position 3 (3T),
(b) isoleucine at position 4 (4I),
(c) alanine, threonine or arginine at position 61 (61A, 61T or 61R),
(d) aspartic acid or glutamic acid at position 154 (154D or 154E),
(e) proline at position 188 (188P),
(f) methionine at position 193 (193M),
(g) isoleucine at position 199 (199I),
(h) aspartic acid, glutamic acid or glycine at position 211 (211D, 211E or 211G),
(i) combinations of amino acids (a) to (h).

The invention also provides the use of a protease selected from the group consisting of
a. protease comprising an amino acid sequence corresponding to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8;
b. protease comprising an amino acid sequence that is modified in at least one position in comparison to SEQ ID NO. 2 or SEQ ID NO. 3 or SEQ ID NO. 4 or SEQ ID NO. 5 or SEQ ID NO. 6 or SEQ ID NO. 7 or SEQ ID NO. 8, the modification in the sequence corresponding to SEQ ID NO. 1 being selected from the group consisting of:
   i. threonine at position 3 (3T),
   ii. isoleucine at position 4 (4I),
   iii. alanine, threonine or arginine at position 61 (61A, 61T or 61R),
   iv. aspartic acid or glutamic acid at position 154 (154D or 154E),
   v. proline at position 188 (188P),
   vi. methionine at position 193 (193M),
   vii. isoleucine at position 199 (199I),
   viii. aspartic acid, glutamic acid or glycine at position 211 (211D, 211E or 211G),
   ix. combinations of amino acids (i) to (viii);
      to provide a proteolytic activity in a liquid washing or cleaning agent that also encompasses a lipase.

All data, subject-matters and embodiments described for washing or cleaning agents according to the invention can also be applied to the specified uses. Therefore reference is expressly made here to the disclosure at the corresponding point, with the note that this disclosure also applies to the above uses according to the invention.

Example: Determining the storage stability of liquid washing agents according to the invention

The following were used as basic washing agent formulations:
a) a first liquid washing agent of the following composition (all figures in percentage by weight): 0.3-0.5% xanthan gum, 0.2-0.4% antifoaming agent, 6-7% glycerol, 0.3-0.5% ethanol, 4-7% FAEOS (fatty alcohol ether sulfate), 24-28% non-ionic surfactants, 1% boric acid, 1-2% sodium citrate (dihydrate), 2-4% sodium carbonate, 14-16% coconut fatty acids, 0.5% HEDP (1-hydroxyethane-(1,1-diphosphonic acid)), 0-0.4% PVP (polyvinylpyrrolidone), 0-0.05% optical brightener, 0-0.001% dye, remainder demineralized water. 0.1 wt. % Lipex 100L (Novozymes, active substance content 1.9%) was included as the lipase.
b) a second liquid washing agent of the following composition:

| Ingredient | wt. % |
|---|---|
| C₁₂₋₁₈ fatty alcohol having 7 EO | 6.40 |
| Linear C₁₀-C₁₃ alkylbenzene sulfonate (Na salt) | 5.35 |
| C₁₂₋₁₈ fatty acid (Na salt) | 2.00 |
| Citric acid (Na salt) | 1.20 |
| Phosphonate (Dequest ® 2066) | 0.50 |
| Boric acid (Na salt) | 1.00 |
| Polyacrylate thickener | 0.15 |
| Glycerol | 3.00 |
| Ethanol | 1.00 |
| Silicone defoaming agent | 0.01 |
| Perfume | 0.70 |
| Dye, preservative | + |
| Water | to 100 |

0.2 wt. % Lipex 100L (Novozymes, active substance content 1.9%) was included as the lipase. For the various experimental batches the following proteases were added to the basic washing agent formulations in identical concentrations, relative to active protein, wherein 0.45 mg of protease (active protein) per gram of washing agent was used in the liquid washing agent according to a) and 0.27 mg of protease (active protein) per gram of washing agent was used in the liquid washing agent according to b): protease comprising an amino acid sequence corresponding to SEQ ID NO. 2 (Glu at position 99 (99E), batch 1), performance-enhanced variant F49 of the protease from Bacillus lentus according to WO 95/23221 (Arg at position 99 (99R), batch 2), and the protease disclosed in FIG. 2 and SEQ ID NO. 3 of the laid-open world patent application WO 03/057713 (Ser at position 99 (99S), identity in relation to SEQ ID NO. 1<80%, batch 3).

The washing agents according to batches 1, 2 and 3 were each tested in terms of their storage stability. To this end the washing agents were stored at a temperature of 30° C. for the specified time in each case and the residual lipolytic activity of each was determined as described in Bruno Stellmach, “Bestimmungsmethoden Enzyme für Pharmazie, Lebensmittelchemie, Technik, Biochemie, Biologie, Medizin” (Steinkopff Verlag Darmstadt, 1988, p. 172 ff): lipase-containing samples were added to an olive oil emulsion in water containing emulsifier and incubated at 30° C. and pH 9.0. Fatty acids were released in the process. These were titrated continuously with 0.01 N sodium hydroxide solution for 20 min with an auto-titrater in such a way that the pH remained constant (pH-stat titration). The lipase activity was determined from the sodium hydroxide consumption by reference to a reference lipase sample. The residual lipolytic activities obtained are set out in Table 1 below.

TABLE 1

| Washing agent | a) | | | b) | | |
|---|---|---|---|---|---|---|
| according to | Start | 4 weeks | 8 weeks | Start | 4 weeks | 8 weeks |
| Batch 1 | 100% | 78% | 64% | 100% | 59% | 38% |
| Batch 2 | 100% | 59% | 38% | 100% | 53% | 34% |
| Batch 3 | 100% | 53% | 25% | 100% | 36% | 10% |

It is clear that washing agents according to the invention have a markedly improved residual lipolytic activity and hence storage stability in comparison to the washing agents from batches 2 and 3.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1-10. (canceled)

11. A method of formulating a washing or cleaning agent for storage stability of lipolytic activity, the method comprising:
(1) mixing, to formulate the washing or cleaning agent:
(a) a protease comprising an amino acid sequence that is at least 80% identical to the amino acid sequence listed in SEQ ID NO: 1 and that has the amino acid glutamic acid (E) at location 99 in a count according to SEQ ID NO: 1; and
(b) a lipase; and
(2) storing the washing or cleaning agent;
wherein the washing or cleaning agent exhibits increased storage stability of lipolytic activity after storage for 4 weeks at 30° C. as compared to a control washing or cleaning agent that differs from the washing or cleaning agent solely by having a protease comprising the amino acid arginine (R) at location 99 in a count according to SEQ ID NO: 1.

12. The method of claim 11, wherein the washing or cleaning agent is stored for at least 5 days.

13. The method of claim 11, wherein the washing or cleaning agent is stored for at least 1 week.

14. The method of claim 11, wherein the washing or cleaning agent is stored for at least 2 weeks.

15. The method of claim 11, wherein the washing or cleaning agent is stored for at least 4 weeks.

16. The method of claim 11, wherein the protease comprises the amino acid sequence of SEQ ID NO: 2.

17. The method of claim 11, wherein the protease comprises the amino acid sequence of SEQ ID NO: 2, wherein the amino acid sequence comprises a changed amino acid sequence in at least one location in SEQ ID NO: 2, wherein the change, in the count according to SEQ ID NO: 1, is selected from the group consisting of:
i. threonine at location 3 (3T),
ii. isoleucine at location 4 (4I),
iii. alanine, threonine or arginine at location 61 (61A, 61T or 61 R),
iv. aspartic acid or glutamic acid at location 154 (154D or 154E),
v. proline at location 188 (188P),
vi. methionine at location 193 (193M),
vii. isoleucine at location 199 (199I),
viii. aspartic acid, glutamic acid or glycine at location 211 (211 D, 211E or 211G), and
ix. a combination thereof.
